(19) United States
(12) Patent Application Publication
(10) Pub. No.: US 2023/0002445 A1
(43) Pub. Date: Jan. 5, 2023

(54) COUPLING ISOELECTRIC FOCUSING-BASED FRACTIONATION WITH MASS SPECTROMETRY ANALYSIS

(71) Applicant: Regeneron Pharmaceuticals, Inc., Tarrytown, NY (US)

(72) Inventors: Tao Xing, White Plains, NY (US); Yuetian Yan, Chappaqua, NY (US); Shunhai Wang, Scarsdale, NY (US)

(21) Appl. No.: 17/853,807

(22) Filed: Jun. 29, 2022

Related U.S. Application Data
(60) Provisional application No. 63/217,125, filed on Jun. 30, 2021.
(60) Provisional application No. 63/301,350, filed on Jan. 20, 2022.

(51) Int. Cl. C07K 1/36; C07K 1/28; C07K 1/22; G01N 33/68; B01D 15/34; B01D 15/16; G01N 30/72

(52) U.S. Cl. CPC C07K 1/36; C07K 1/28; C07K 1/22; G01N 33/6848; B01D 15/34; B01D 15/168; G01N 30/72

(57) ABSTRACT

The present invention generally pertains to methods of characterizing charge variants of a protein of interest. In particular, the present invention pertains to the use of desalting size exclusion chromatography-reduced peptide mapping mass spectrometry to identify charge variants separated by capillary isoelectric focusing.

### CROSS-REFERENCE TO RELATED APPLICATIONS

This application claims priority to and the benefit of U.S. Provisional Patent Application No. 63/217,125, filed Jun. 30, 2021 and U.S. Provisional Patent Application No. 63/301,350, filed Jan. 20, 2022 which are each herein incorporated by reference.

### FIELD

This application relates to methods for characterization of charge variants of therapeutic proteins.

### BACKGROUND

Biophysical properties, including domain-specific variants, of therapeutic peptides and proteins can affect their safety, efficacy and shelf-life. For example, the presence of different charge variants may alter protein solubility, binding, and stability.

Therapeutic peptides or proteins, such as antibodies, may acquire different variants and become heterogeneous due to various post-translation modifications (PTMs), protein degradation, enzymatic modifications, and chemical modifications. These alterations to biophysical properties may occur at almost any point during and after peptide and protein production. Because these alterations to biophysical characteristics may affect the safety, efficacy, and shelf-life of therapeutic peptides and proteins, it is important to identify different variants for particular therapeutic peptides or proteins, and furthermore to interrogate the modifications responsible for charge variants.

Isoelectric focusing (IEF) has become a common tool for separating components of a sample based on charge (pI), thus allowing for the separation of charge variants of a protein. IEF analysis may also be combined with mass spectrometry (MS) analysis to gain further information on the protein associated with each charge variant. However, conventional methods are limited in the techniques that can be used in IEF-MS analysis. To date, it has not been possible to perform reduced peptide mapping analysis of high-resolution, narrow IEF fractions. Thus, it has not been possible to identify the site, for example the amino acid residue, of specific protein modifications associated with specific charge variants.

Therefore, it will be appreciated that a need exists for methods and systems to specifically characterize modifications of a therapeutic protein associated with charge variants.

### SUMMARY

A method has been developed for characterization of charge variants of a protein of interest. In an exemplary embodiment, a sample including a protein of interest is subjected to capillary isoelectric focusing analysis. A UV trace of the protein sample is generated, which includes UV peaks corresponding to charge variants. Fractions of the sample are collected after isoelectric focusing. Fractions may be collected in a high-throughput fashion such that they represent the entire sample output from the isoelectric focusing step and may comprise narrow intervals, for example, 15 second intervals. Fractions are further processed using desalting size exclusion chromatography, which separates analytes by size in addition to modifying the fraction buffer to be compatible with mass spectrometry analysis. Finally, the eluate from desalting size exclusion chromatography is subjected to mass spectrometry analysis, which can be used to identify specific post-translational modifications corresponding to each fraction and thus to each charge variant.

This disclosure provides a method for characterization of charge variants of a protein of interest. In some exemplary embodiments, the method comprises: (a) subjecting a sample including a protein of interest to capillary isoelectric focusing to separate charge variants of said protein of interest; (b) collecting fractions from said capillary isoelectric focusing step; (c) subjecting said fractions to desalting size exclusion chromatography; and (d) subjecting the eluate from step (c) to mass spectrometry analysis to characterize said charge variants of said protein of interest.

In one aspect, said protein is an antibody, a bispecific antibody, a monoclonal antibody, a fusion protein, an antibody-drug conjugate, an antibody fragment, or a protein pharmaceutical product. In another aspect, said capillary isoelectric focusing is imaged capillary isoelectric focusing. In yet another aspect, said desalting size exclusion chromatography system is coupled to said mass spectrometer.

In one aspect, said mass spectrometer is an electrospray ionization mass spectrometer, nano-electrospray ionization mass spectrometer, or a triple quadrupole mass spectrometer. In another aspect, said mass spectrometry analysis comprises intact mass analysis or reduced peptide mapping analysis. In yet another aspect, said mass spectrometer is capable performing a multiple reaction monitoring or parallel reaction monitoring.

In one aspect, the method further comprises a step wherein said fractions are contacted to at least one hydrolyzing agent prior to desalting size-exclusion chromatography. In a specific aspect, said at least one hydrolyzing agent is chosen from a group consisting of trypsin, chymotrypsin, LysC, LysN, AspN, GluC and ArgC.

In one aspect, the method further comprises a step wherein said fractions are contacted to at least one reducing agent prior to desalting size-exclusion chromatography. In another aspect, said desalting size exclusion chromatography is performed under native conditions.

These, and other, aspects of the invention will be better appreciated and understood when considered in conjunction with the following description and accompanying drawings. The following description, while indicating various embodiments and numerous specific details thereof, is given by way of illustration and not of limitation. Many substitutions, modifications, additions, or rearrangements may be made within the scope of the invention.

### BRIEF DESCRIPTION OF THE DRAWINGS

FIG. 1 illustrates a workflow of the method of the invention according to an exemplary embodiment.

FIG. 2A shows a correlation of peaks from a UV trace detected by capillary isoelectric focusing (cIEF) and corresponding peaks from desalting size exclusion chromatography-mass spectrometry (SEC-MS) analysis according to an exemplary embodiment.

FIG. 2B shows charge variants identified by desalting SEC-MS and corresponding cIEF peaks and fractions according to an exemplary embodiment.

FIG. 3A shows a deconvoluted mass spectrum of charge variants detected by desalting SEC-MS corresponding to the main UV peak detected by cIEF according to an exemplary embodiment.

FIG. 3B shows a deconvoluted mass spectrum of charge variants detected by desalting SEC-MS corresponding to the B1 UV peak detected by cIEF according to an exemplary embodiment.

FIG. 3C shows a deconvoluted mass spectrum of charge variants detected by desalting SEC-MS corresponding to the B2 UV peak detected by cIEF according to an exemplary embodiment.

FIG. 3D shows a deconvoluted mass spectrum of charge variants detected by desalting SEC-MS corresponding to the A1 UV peak detected by cIEF according to an exemplary embodiment.

FIG. 3E shows a deconvoluted mass spectrum of charge variants detected by desalting SEC-MS corresponding to the A2 UV peak detected by cIEF according to an exemplary embodiment.

FIG. 3F shows a deconvoluted mass spectrum of charge variants detected by desalting SEC-MS corresponding to the A3 UV peak detected by cIEF according to an exemplary embodiment.

FIG. 4 shows reduced peptide mapping spectra for each UV peak detected by cIEF according to an exemplary embodiment.

FIG. 5A shows the distribution of aspartic acid isomerization at multiple amino acid residues for each UV peak detected by cIEF according to an exemplary embodiment.

FIG. 5B shows the distribution of aspartic acid cyclization at multiple amino acid residues for each UV peak detected by cIEF according to an exemplary embodiment.

FIG. 5C shows the distribution of asparagine deamidation at multiple amino acid residues for each UV peak detected by cIEF according to an exemplary embodiment.

FIG. 5D shows the distribution of asparagine succinimides at multiple amino acid residues for each UV peak detected by cIEF according to an exemplary embodiment.

FIG. 5E shows the distribution of lysine glycation at multiple amino acid residues for each UV peak detected by cIEF according to an exemplary embodiment.

FIG. 5F shows the distribution of C-terminal lysines at each heavy chain for each UV peak detected by cIEF according to an exemplary embodiment.

FIG. 5G shows the distribution of N-acetylneuraminic acid for each UV peak detected by cIEF according to an exemplary embodiment.

### DETAILED DESCRIPTION

Therapeutic antibodies produced in mammalian cells, including monoclonal antibodies (mAbs) or bispecific antibodies (bsAbs), are heterogeneous as a result of post-translational modifications (PTMs), enzymatic modifications and chemical modifications, which contribute to size and charge variants. These modifications may include, for example, glycosylation, deglycosylation, amidation, deamidation, oxidation, glycation, terminal cyclization, C-terminal lysine variation, C-terminal arginine variation, N-terminal pyroglutamate variation, C-terminal glycine amidation, C-terminal proline amidation, succinimide formation, sialylation, or desialylation. In addition, aggregation, degradation, denaturation, fragmentation, or isomerization of protein products can also introduced charge heterogeneity. Table 1 shows exemplary protein modifications and their impacts on changing the electric charges of peptides or proteins.

TABLE 1
Exemplary protein modifications that may cause charge variants

| Protein modifications | Effect | Species formed |
|---|---|---|
| Sialylation | COOH addition | Acidic |
| Deamidation | COOH formation | Acidic |
| C-terminal lysine cleavage | Loss of NH2 | Acidic |
| Adduct formation | COOH formation | Acidic |
| | or loss of NH2 | |
| Succinimide formation | Loss of COOH | Basic |
| Methionine, cysteine, lysine, | Conformational | Basic |
| histidine, tryptophan oxidation | change | |
| Asialylation (terminal galactose) | Loss of COOH | Basic |
| C-terminal lysine and glycine | NH2 formation | Basic |
| amidation | or loss of COOH | |

During the manufacture of a therapeutic peptide or protein, such as a monoclonal antibody, charge heterogeneity is potentially introduced as a result of protein degradation and/or the presence of PTMs. Characterization of charge variant forms of a protein within the manufactured drug substance is required to fully understand the correlation between properties of the protein, such as potency, and the physical and chemical changes associated with the charge variants.

Several methods exist that allow for separation of protein charge variants, including ion exchange chromatography and isoelectric focusing (IEF). IEF has become a more common approach because of its capacity for high-resolution separation of sample components based on pI, and its ability to take into account both surface-exposed and internal amino acids with no loss of resolution due to hydrophobic interactions. IEF, particularly capillary IEF (cIEF), can also be combined with mass spectrometry (MS) analysis to gain further information on a protein sample. However, the buffers used for cIEF and MS are not immediately compatible, which creates difficulties in using the sample separated by cIEF for MS analysis. Two main approaches have been taken to solve this issue, using either offline or online connection to MS.

When using an offline connection, fractions from cIEF are collected, the buffer is modified to be compatible with MS, and the modified fractions are subjected to MS analysis. However, fraction collection from cIEF has been low-throughput, resulting in few, large fractions being collected, which reduces the resolution and specificity of charge variant analysis.

Alternatively, an online connection can be used, outputting the separated sample from cIEF into MS without a fraction collection step. This requires intermediate online steps such as interim chromatography or dialysis to modify the sample buffer between cIEF and MS analysis. While this online connection preserves the high resolution separation of cIEF, it does not allow for alternative processing of cIEF-separated samples, for example digestion or reduction of a protein of interest, and thus is limited to intact mass analysis.

Thus, there exists a need for methods and systems to characterize charge variants of a protein of interest in a flexible and high resolution manner. Particularly, there exists a need for a method to identify site-specific protein modifications associated with charge variants.

This disclosure sets forth a novel method to identify site-specific protein modifications associated with charge variants of a protein of interest. The method employs cIEF with an offline connection to MS. Unlike previous approaches, cIEF fractions are collected in a comprehensive and high-throughput fashion, for example collecting all output from a cIEF capillary, separated into fractions each representing a 15 second interval. This novel high-throughput fraction collection allows for offline processing of cIEF-separated samples with no substantive loss of pI resolution. Collected fractions may be further processed, for example, by contacting them with hydrolytic agents and/or reducing agents to produce reduced peptides for reduced peptide mapping analysis. Collected fractions may be subjected to a variety of processing steps according to the needs of the user, or no processing steps if used, for example, for intact mass analysis.

Collected fractions are then individually subjected to desalting size exclusion chromatography (SEC), as described for example in Yan et al., 2020, J Am Soc Mass Spectrom, 31:2171-2179. This high-throughput desalting SEC method allows for efficient processing of fractions, further separating sample components by size as well as modifying the fraction buffer to be compatible with MS. The desalting SEC system may be connected online to a mass spectrometer.

Fractions output from desalting SEC are subjected to MS analysis, for example intact mass analysis or reduced peptide mapping analysis. MS analysis creates fragments of analytes and separates them based on mass-to-charge (m z) ratio. This separation allows for the identification of modifications to the protein, such as PTMs. In particular, the resolution of reduced peptide mapping analysis allows for site-specific identification of PTMs, for example identifying a specific chemical change at a specific amino acid residue on the protein of interest. An identified site-specific modification, arising from a known cIEF fraction, can then be associated with a specific charge variant of the protein of interest. This method allows for high-resolution, high-throughput analysis of site-specific protein modifications that give rise to charge variants, allowing for monitoring and improvement of therapeutic protein production processes in order to validate and optimize protein biophysical characteristics and homogeneity.

Unless described otherwise, all technical and scientific terms used herein have the same meaning as commonly understood by one of ordinary skill in the art to which this invention belongs. Although any methods and materials similar or equivalent to those described herein can be used in the practice or testing, particular methods and materials are now described.

The term “a” should be understood to mean “at least one” and the terms “about” and “approximately” should be understood to permit standard variation as would be understood by those of ordinary skill in the art, and where ranges are provided, endpoints are included. As used herein, the terms “include,” “includes,” and “including” are meant to be non-limiting and are understood to mean “comprise,” “comprises,” and “comprising” respectively.

As used herein, the term “protein” or “protein of interest” can include any amino acid polymer having covalently linked amide bonds. Proteins comprise one or more amino acid polymer chains, generally known in the art as “polypeptides.” “Polypeptide” refers to a polymer composed of amino acid residues, related naturally occurring structural variants, and synthetic non-naturally occurring analogs thereof linked via peptide bonds. “Synthetic peptide or polypeptide” refers to a non-naturally occurring peptide or polypeptide. Synthetic peptides or polypeptides can be synthesized, for example, using an automated polypeptide synthesizer. Various solid phase peptide synthesis methods are known to those of skill in the art. A protein may comprise one or multiple polypeptides to form a single functioning biomolecule. In another exemplary aspect, a protein can include antibody fragments, nanobodies, recombinant antibody chimeras, cytokines, chemokines, peptide hormones, and the like. Proteins of interest can include any of bio-therapeutic proteins, recombinant proteins used in research or therapy, trap proteins and other chimeric receptor Fc-fusion proteins, chimeric proteins, antibodies, monoclonal antibodies, polyclonal antibodies, human antibodies, and bispecific antibodies. Proteins may be produced using recombinant cell-based production systems, such as the insect bacculovirus system, yeast systems (e.g., Pichia sp.), and mammalian systems (e.g., CHO cells and CHO derivatives like CHO-K1 cells). For a recent review discussing biotherapeutic proteins and their production, see Ghaderi et al., “Production platforms for biotherapeutic glycoproteins. Occurrence, impact, and challenges of non-human sialylation” (Darius Ghaderi et al., Production platforms for biotherapeutic glycoproteins. Occurrence, impact, and challenges of non-human sialylation, 28 BIOTECHNOLOGY AND GENETIC ENGINEERING REVIEWS 147-176 (2012), the entire teachings of which are herein incorporated). In some exemplary embodiments, proteins comprise modifications, adducts, and other covalently linked moieties. These modifications, adducts and moieties include, for example, avidin, streptavidin, biotin, glycans (e.g., N-acetylgalactosamine, galactose, neuraminic acid, N-acetylglucosamine, fucose, mannose, and other monosaccharides), PEG, polyhistidine, FLAGtag, maltose binding protein (MBP), chitin binding protein (CBP), glutathione-S-transferase (GST) myc-epitope, fluorescent labels and other dyes, and the like. Proteins can be classified on the basis of compositions and solubility and can thus include simple proteins, such as globular proteins and fibrous proteins; conjugated proteins, such as nucleoproteins, glycoproteins, mucoproteins, chromoproteins, phosphoproteins, metalloproteins, and lipoproteins; and derived proteins, such as primary derived proteins and secondary derived proteins.

In some exemplary embodiments, the protein of interest can be a recombinant protein, an antibody, a bispecific antibody, a multispecific antibody, antibody fragment, monoclonal antibody, fusion protein, scFv and combinations thereof.

As used herein, the term “recombinant protein” refers to a protein produced as the result of the transcription and translation of a gene carried on a recombinant expression vector that has been introduced into a suitable host cell. In certain exemplary embodiments, the recombinant protein can be an antibody, for example, a chimeric, humanized, or fully human antibody. In certain exemplary embodiments, the recombinant protein can be an antibody of an isotype selected from group consisting of. IgG, IgM, IgA1, IgA2, IgD, or IgE. In certain exemplary embodiments the antibody molecule is a full-length antibody (e.g., an IgG1) or alternatively the antibody can be a fragment (e.g., an Fc fragment or a Fab fragment).

The term “antibody,” as used herein includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or VH) and a heavy chain constant region. The heavy chain constant region comprises three domains, CH1, CH2 and CH3. Each light chain comprises a light chain variable region (abbreviated herein as LCVR or VL) and a light chain constant region. The light chain constant region comprises one domain (CL1). The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, and FR4. In different embodiments of the invention, the FRs of the anti-big-ET-1 antibody (or antigen-binding portion thereof) may be identical to the human germline sequences or may be naturally or artificially modified. An amino acid consensus sequence may be defined based on a side-by-side analysis of two or more CDRs. The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, for example, from full antibody molecules using any suitable standard techniques such as proteolytic digestion or recombinant genetic engineering techniques involving the manipulation and expression of DNA encoding antibody variable and optionally constant domains. Such DNA is known and/or is readily available from, for example, commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

As used herein, an “antibody fragment” includes a portion of an intact antibody, such as, for example, the antigen-binding or variable region of an antibody. Examples of antibody fragments include, but are not limited to, a Fab fragment, a Fab′ fragment, a F(ab′)2 fragment, a scFv fragment, a Fv fragment, a dsFv diabody, a dAb fragment, a Fd′ fragment, a Fd fragment, and an isolated complementarity determining region (CDR) region, as well as triabodies, tetrabodies, linear antibodies, single-chain antibody molecules, and multi specific antibodies formed from antibody fragments. Fv fragments are the combination of the variable regions of the immunoglobulin heavy and light chains, and ScFv proteins are recombinant single chain polypeptide molecules in which immunoglobulin light and heavy chain variable regions are connected by a peptide linker. In some exemplary embodiments, an antibody fragment comprises a sufficient amino acid sequence of the parent antibody of which it is a fragment that it binds to the same antigen as does the parent antibody; in some exemplary embodiments, a fragment binds to the antigen with a comparable affinity to that of the parent antibody and/or competes with the parent antibody for binding to the antigen. An antibody fragment may be produced by any means. For example, an antibody fragment may be enzymatically or chemically produced by fragmentation of an intact antibody and/or it may be recombinantly produced from a gene encoding the partial antibody sequence. Alternatively, or additionally, an antibody fragment may be wholly or partially synthetically produced. An antibody fragment may optionally comprise a single chain antibody fragment. Alternatively, or additionally, an antibody fragment may comprise multiple chains that are linked together, for example, by disulfide linkages. An antibody fragment may optionally comprise a multi-molecular complex. A functional antibody fragment typically comprises at least about 50 amino acids and more typically comprises at least about 200 amino acids.

The term “bispecific antibody” includes an antibody capable of selectively binding two or more epitopes. Bispecific antibodies generally comprise two different heavy chains with each heavy chain specifically binding a different epitope-either on two different molecules (e.g., antigens) or on the same molecule (e.g., on the same antigen). If a bispecific antibody is capable of selectively binding two different epitopes (a first epitope and a second epitope), the affinity of the first heavy chain for the first epitope will generally be at least one to two or three or four orders of magnitude lower than the affinity of the first heavy chain for the second epitope, and vice versa. The epitopes recognized by the bispecific antibody can be on the same or a different target (e.g., on the same or a different protein). Bispecific antibodies can be made, for example, by combining heavy chains that recognize different epitopes of the same antigen. For example, nucleic acid sequences encoding heavy chain variable sequences that recognize different epitopes of the same antigen can be fused to nucleic acid sequences encoding different heavy chain constant regions and such sequences can be expressed in a cell that expresses an immunoglobulin light chain.

A typical bispecific antibody has two heavy chains each having three heavy chain CDRs, followed by a CH1 domain, a hinge, a CH2 domain, and a CH3 domain, and an immunoglobulin light chain that either does not confer antigen-binding specificity but that can associate with each heavy chain, or that can associate with each heavy chain and that can bind one or more of the epitopes bound by the heavy chain antigen-binding regions, or that can associate with each heavy chain and enable binding of one or both of the heavy chains to one or both epitopes. BsAbs can be divided into two major classes, those bearing an Fc region (IgG-like) and those lacking an Fc region, the latter normally being smaller than the IgG and IgG-like bispecific molecules comprising an Fc. The IgG-like bsAbs can have different formats such as, but not limited to, triomab, knobs into holes IgG (kih IgG), crossMab, orth-Fab IgG, Dual-variable domains Ig (DVD-Ig), two-in-one or dual action Fab (DAF), IgG-single-chain Fv (IgG-scFv), or κλ-bodies. The non-IgG-like different formats include tandem scFvs, diabody format, single-chain diabody, tandem diabodies (TandAbs), Dual-affinity retargeting molecule (DART), DART-Fc, nanobodies, or antibodies produced by the dock-and-lock (DNL) method (Gaowei Fan, Zujian Wang & Mingju Hao, Bispecific antibodies and their applications, 8 JOURNAL OF HEMATOLOGY & ONCOLOGY 130; Dafne Müller & Roland E. Kontermann, Bispecific Antibodies, HANDBOOK OF THERAPEUTIC ANTIBODIES 265-310 (2014), the entire teachings of which are herein incorporated). The methods of producing bsAbs are not limited to quadroma technology based on the somatic fusion of two different hybridoma cell lines, chemical conjugation, which involves chemical cross-linkers, and genetic approaches utilizing recombinant DNA technology. Examples of bsAbs include those disclosed in the following patent applications, which are hereby incorporated by reference: U.S. Ser. No. 12/823,838, filed Jun. 25, 2010; U.S. Ser. No. 13/488,628, filed Jun. 5, 2012; U.S. Ser. No. 14/031,075, filed Sep. 19, 2013; U.S. Ser. No. 14/808,171, filed Jul. 24, 2015; U.S. Ser. No. 15/713,574, filed Sep. 22, 2017; U.S. Ser. No. 15/713,569, field Sep. 22, 2017; U.S. Ser. No. 15/386,453, filed Dec. 21, 2016; U.S. Ser. No. 15/386,443, filed Dec. 21, 2016; U.S. Ser. No. 15/22,343 filed Jul. 29, 2016; and U.S. Ser. No. 15/814,095, filed Nov. 15, 2017.

As used herein “multispecific antibody” refers to an antibody with binding specificities for at least two different antigens. While such molecules normally will only bind two antigens (i.e., bispecific antibodies, bsAbs), antibodies with additional specificities such as trispecific antibody and KIH Trispecific can also be addressed by the system and method disclosed herein.

The term “monoclonal antibody” as used herein is not limited to antibodies produced through hybridoma technology. A monoclonal antibody can be derived from a single clone, including any eukaryotic, prokaryotic, or phage clone, by any means available or known in the art. Monoclonal antibodies useful with the present disclosure can be prepared using a wide variety of techniques known in the art including the use of hybridoma, recombinant, and phage display technologies, or a combination thereof.

In some exemplary embodiments, the protein of interest can have a pI in the range of about 4.5 to about 9.0. In one exemplary specific embodiment, the pI can be about 4.5, about 5.0, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, about 8.0, about 8.1, about 8.2, about 8.3, about 8.4, about 8.5, about 8.6, about 8.7, about 8.8, about 8.9, or about 9.0. In some exemplary embodiments, the types of protein of interest in the compositions can be more than one.

In some exemplary embodiments, the protein of interest can be produced from mammalian cells. The mammalian cells can be of human origin or non-human origin can include primary epithelial cells (e.g., keratinocytes, cervical epithelial cells, bronchial epithelial cells, tracheal epithelial cells, kidney epithelial cells and retinal epithelial cells), established cell lines and their strains (e.g., 293 embryonic kidney cells, BHK cells, HeLa cervical epithelial cells and PER-C6 retinal cells, MDBK (NBL-1) cells, 911 cells, CRFK cells, MDCK cells, CHO cells, BeWo cells, Chang cells, Detroit 562 cells, HeLa 229 cells, HeLa S3 cells, Hep-2 cells, KB cells, LSI80 cells, LS174T cells, NCI-H-548 cells, RPM12650 cells, SW-13 cells, T24 cells, WI-28 VA13, 2RA cells, WISH cells, BS-C-I cells, LLC-MK2 cells, Clone M-3 cells, 1-10 cells, RAG cells, TCMK-1 cells, Y-1 cells, LLC-PKi cells, PK(15) cells, GHi cells, GH3 cells, L2 cells, LLC-RC 256 cells, MHiCi cells, XC cells, MDOK cells, VSW cells, and TH-I, B1 cells, BSC-1 cells, RAf cells, RK-cells, PK-15 cells or derivatives thereof), fibroblast cells from any tissue or organ (including but not limited to heart, liver, kidney, colon, intestines, esophagus, stomach, neural tissue (brain, spinal cord), lung, vascular tissue (artery, vein, capillary), lymphoid tissue (lymph gland, adenoid, tonsil, bone marrow, and blood), spleen, and fibroblast and fibroblast-like cell lines (e.g., CHO cells, TRG-2 cells, IMR-33 cells, Don cells, GHK-21 cells, citrullinemia cells, Dempsey cells, Detroit 551 cells, Detroit 510 cells, Detroit 525 cells, Detroit 529 cells, Detroit 532 cells, Detroit 539 cells, Detroit 548 cells, Detroit 573 cells, HEL 299 cells, IMR-90 cells, MRC-5 cells, WI-38 cells, WI-26 cells, Midi cells, CHO cells, CV-1 cells, COS-1 cells, COS-3 cells, COS-7 cells, Vero cells, DBS-FrhL-2 cells, BALB/3T3 cells, F9 cells, SV-T2 cells, M-MSV-BALB/3T3 cells, K-BALB cells, BLO-11 cells, NOR-10 cells, C3H/IOTI/2 cells, HSDMiC3 cells, KLN205 cells, McCoy cells, Mouse L cells, Strain 2071 (Mouse L) cells, L-M strain (Mouse L) cells, L-MTK′ (Mouse L) cells, NCTC clones 2472 and 2555, SCC-PSA1 cells, Swiss/3T3 cells, Indian muntjac cells, SIRC cells, Cn cells, and Jensen cells, Sp2/0, NS0, NS1 cells or derivatives thereof).

In some exemplary embodiments, the sample including the protein of interest can be prepared prior to desalting SEC-MS analysis. Preparation steps can include alkylation, reduction, denaturation, and/or digestion.

As used herein, the term “protein alkylating agent” refers to an agent used for alkylating certain free amino acid residues in a protein. Non-limiting examples of protein alkylating agents are iodoacetamide (IOA), chloroacetamide (CAA), acrylamide (AA), N-ethylmaleimide (NEM), methyl methanethiosulfonate (MMTS), and 4-vinylpyridine or combinations thereof.

As used herein, “protein denaturing” can refer to a process in which the three-dimensional shape of a molecule is changed from its native state. Protein denaturation can be carried out using a protein denaturing agent. Non-limiting examples of a protein denaturing agent include heat, high or low pH, reducing agents like DTT (see below) or exposure to chaotropic agents. Several chaotropic agents can be used as protein denaturing agents. Chaotropic solutes increase the entropy of the system by interfering with intramolecular interactions mediated by non-covalent forces such as hydrogen bonds, van der Waals forces, and hydrophobic effects. Non-limiting examples for chaotropic agents include butanol, ethanol, guanidinium chloride, lithium perchlorate, lithium acetate, magnesium chloride, phenol, propanol, sodium dodecyl sulfate, thiourea, N-lauroylsarcosine, urea, and salts thereof.

As used herein, the term “protein reducing agent” refers to the agent used for reduction of disulfide bridges in a protein. Non-limiting examples of protein reducing agents used to reduce a protein are dithiothreitol (DTT), ß-mercaptoethanol, Ellman's reagent, hydroxylamine hydrochloride, sodium cyanoborohydride, tris(2-carboxyethyl)phosphine hydrochloride (TCEP-HCl), or combinations thereof.

As used herein, the term “digestion” refers to hydrolysis of one or more peptide bonds of a protein. There are several approaches to carrying out digestion of a protein in a sample using an appropriate hydrolyzing agent, for example, enzymatic digestion or non-enzymatic digestion.

As used herein, the term “digestive enzyme” refers to any of a large number of different agents that can perform digestion of a protein. Non-limiting examples of hydrolyzing agents that can carry out enzymatic digestion include protease from Aspergillus saitoi, elastase, subtilisin, protease XIII, pepsin, trypsin, Tryp-N, chymotrypsin, aspergillopepsin I, LysN protease (Lys-N), LysC endoproteinase (Lys-C), endoproteinase Asp-N (Asp-N), endoproteinase Arg-C (Arg-C), endoproteinase Glu-C (Glu-C) or outer membrane protein T (OmpT), immunoglobulin-degrading enzyme of Streptococcus pyogenes (IdeS), thermolysin, papain, pronase, V8 protease or biologically active fragments or homologs thereof or combinations thereof. For a recent review discussing the available techniques for protein digestion see Switazar et al., “Protein Digestion: An Overview of the Available Techniques and Recent Developments” (Linda Switzar, Martin Giera & Wilfried M. A. Niessen, Protein Digestion: An Overview of the Available Techniques and Recent Developments, 12 JOURNAL OF PROTEOME RESEARCH 1067-1077 (2013)).

As used herein, the term “charge variant” or “variant” of a polypeptide refers to a polypeptide comprising an amino acid sequence that is at least about 70-99.9% (e.g., 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.5, 99.9%) identical or similar to a referenced or native amino acid sequence of a protein of interest. A sequence comparison can be performed by, for example, a BLAST algorithm wherein the parameters of the algorithm are selected to give the largest match between the respective sequences over the entire length of the respective reference sequences (e.g., expect threshold: 10; word size: 3; max matches in a query range: 0; BLOSUM 62 matrix; gap costs: existence 11, extension 1; conditional compositional score matrix adjustment). Variants of a polypeptide may also refer to a polypeptide comprising a referenced amino acid sequence except for one or more (e.g., 1, 2, 3, 4, 5, 6, 7, 8, 9 or 10) mutations such as, for example, missense mutations (e.g., conservative substitutions), nonsense mutations, deletions, or insertions. The following references relate to BLAST algorithms often used for sequence analysis: BLAST ALGORITHMS: Altschul et al. (2005) FEBS J. 272(20): 5101-5109; Altschul, S. F., et al., (1990) J. Mol. Biol. 215:403-410; Gish, W., et al., (1993) Nature Genet. 3:266-272; Madden, T. L., et al., (1996) Meth. Enzymol. 266:131-141; Altschul, S. F., et al., (1997) Nucleic Acids Res. 25:3389-3402; Zhang, J., et al., (1997) Genome Res. 7:649-656; Wootton, J. C., et al., (1993) Comput. Chem. 17:149-163; Hancock, J. M. et al., (1994) Comput. Appl. Biosci. 10:67-70; ALIGNMENT SCORING SYSTEMS: Dayhoff, M. O., et al., “A model of evolutionary change in proteins.” in Atlas of Protein Sequence and Structure, (1978) vol. 5, suppl. 3. M. O. Dayhoff (ed.), pp. 345-352, Natl. Biomed. Res. Found., Washington, D.C.; Schwartz, R. M., et al., “Matrices for detecting distant relationships.” in Atlas of Protein Sequence and Structure, (1978) vol. 5, suppl. 3.” M. O. Dayhoff (ed.), pp. 353-358, Natl. Biomed. Res. Found., Washington, D.C.; Altschul, S. F., (1991) J. Mol. Biol. 219:555-565; States, D. J., et al., (1991) Methods 3:66-70; Henikoff, S., et al., (1992) Proc. Natl. Acad. Sci. USA 89:10915-10919; Altschul, S. F., et al., (1993) J. Mol. Evol. 36:290-300; ALIGNMENT STATISTICS: Karlin, S., et al., (1990) Proc. Natl. Acad. Sci. USA 87:2264-2268; Karlin, S., et al., (1993) Proc. Natl. Acad. Sci. USA 90:5873-5877; Dembo, A., et al., (1994) Ann. Prob. 22:2022-2039; and Altschul, S. F. “Evaluating the statistical significance of multiple distinct local alignments.” in Theoretical and Computational Methods in Genome Research (S. Suhai, ed.), (1997) pp. 1-14, Plenum, N.Y.; the entire teachings of which are herein incorporated.

Some variants can be covalent modifications that polypeptides undergo, either during (co-translational modification) or after (post-translational modification “PTM”) their ribosomal synthesis. PTMs are generally introduced by specific enzymes or enzyme pathways. Many occur at the site of a specific characteristic protein sequence (e.g., signature sequence) within the protein backbone. Several hundred PTMs have been recorded and these modifications invariably influence some aspect of a protein's structure or function (Walsh, G. “Proteins” (2014) second edition, published by Wiley and Sons, Ltd., ISBN: 9780470669853, the entire teachings of which are herein incorporated).

In certain exemplary embodiments, a protein composition can comprise more than one type of variant of a protein of interest. Such variants can include both acidic species and basic species. Acidic species are typically the variants that elute earlier than the main peak from CEX or later than the main peak from AEX, while basic species are the variants that elute later than the main peak from CEX or earlier than the main peak from AEX. In an exemplary embodiment, basic species may elute earlier than the main peak from cIEF and acidic species may elute later than the main peak from cIEF.

As used herein, the terms “acidic species,” “AS,” “acidic region,” and “AR,” refer to the variants of a protein which are characterized by an overall acidic charge.

In certain embodiments, the sample can comprise more than one type of acidic species variant. For example, but not by way of limitation, the total acidic species can be categorized based on chromatographic retention time of the peaks appearing, or by UV peaks generated using IEF.

Among the chemical degradation pathways responsible for acidic or basic species, the two most commonly observed covalent modifications occurring in proteins and peptides are deamination and oxidation. Methionine, cysteine, histidine, tryptophan, and tyrosine are some of the amino acids that are most susceptible to oxidation: Met and Cys because of their sulfur atoms and His, Trp, and Tyr because of their aromatic rings.

As used herein, the terms “oxidative species,” “OS,” or “oxidation variant” refer to the variants of a protein formed by oxidation. Such oxidative species can also be detected by various methods, such as ion exchange, for example, WCX-10 HPLC (a weak cation exchange chromatography), or IEF. Oxidation variants can result from oxidation occurring at histidine, cysteine, methionine, tryptophan, phenylalanine and/or tyrosine residues.

As used herein, the terms “basic species,” “basic region,” and “BR,” refer to the variants of a protein, for example, an antibody or antigen-binding portion thereof, which are characterized by an overall basic charge, relative to the primary charge variant species present within the protein. For example, in recombinant protein preparations, such basic species can be detected by various methods, such as ion exchange, for example, WCX-10 HPLC (a weak cation exchange chromatography), or IEF. Exemplary variants can include, but are not limited to, lysine variants, isomerization of aspartic acid, succinimide formation at asparagine, methionine oxidation, amidation, incomplete disulfide bond formation, mutation from serine to arginine, aglycosylation, fragmentation and aggregation. Commonly, basic species elute later than the main peak during CEX or earlier than the main peak during AEX analysis. (Chromatographic analysis of the acidic and basic species of recombinant monoclonal antibodies. MAbs. 2012 Sep. 1; 4(5): 578-585. doi: 10.4161/mabs.21328, the entire teaching of which is herein incorporated by reference.)

In certain embodiments, the sample can comprise more than one type of basic species variant. For example, but not by way of limitation, the total basic species can be divided based on chromatographic retention time of the peaks appearing, or based on UV peaks generated using IEF. Another example in which the total basic species can be divided can be based on the type of variant—variants, structure variants, or fragmentation variant.

As used herein, “sample” can be obtained from any step of the bioprocess, such as cell culture fluid (CCF), harvested cell culture fluid (HCCF), any step in the downstream processing, drug substance (DS), or a drug product (DP) comprising the final formulated product. In some other specific exemplary embodiments, the sample can be selected from any step of the downstream process of clarification, chromatographic production, viral inactivation, or filtration. In some specific exemplary embodiments, the drug product can be selected from manufactured drug product in the clinic, shipping, storage, or handling.

In some aspects, the method disclosed can include subjecting the sample to a capillary isoelectric focusing to separate charge variants of said protein of interest.

As used herein, “isoelectric focusing” or “IEF”, also known simply as electrofocusing, is a technique for separating charged molecules, usually proteins or peptides, on the basis of their isoelectric point (pI), for example, the pH at which the molecule has no charge. IEF works because in an electric field molecules in a pH gradient will migrate towards their pI. A variety of techniques for conducting IEF exist. For example, in capillary isoelectric focusing (cIEF), samples travel through a capillary based on an applied electric field. A UV detector may be used at a point along the capillary to detect the time at which an analyte, such as a protein, traverses that point of the capillary. Because travel time through the capillary is directly related to the charge (pI) of the analyte, UV signal from a point in the capillary over time can be represented as a UV trace, which represents the varying charges (pI) of sample components. In an exemplary embodiment, a UV trace generated by cIEF represents charge variants of a protein of interest, with each UV peak representing a significant charge variant. Variations of cIEF may also be used, for example, imaged cIEF (icIEF).

Size exclusion chromatography (SEC) or gel filtration relies on the separation of components as a function of their molecular size. Separation depends on the amount of time that the substances spend in the porous stationary phase as compared to time in the fluid. The probability that a molecule will reside in a pore depends on the size of the molecule and the pore. In addition, the ability of a substance to permeate into pores is determined by the diffusion mobility of macromolecules which is higher for small macromolecules. Very large macromolecules may not penetrate the pores of the stationary phase at all; and, for very small macromolecules the probability of penetration is close to unity. While components of larger molecular size move more quickly past the stationary phase, components of small molecular size have a longer path length through the pores of the stationary phase and are thus retained longer in the stationary phase.

The chromatographic material can comprise a size exclusion material wherein the size exclusion material is a resin or membrane. The matrix used for size exclusion is preferably an inert gel medium which can be a composite of cross-linked polysaccharides, for example, cross-linked agarose and/or dextran in the form of spherical beads. The degree of cross-linking determines the size of pores that are present in the swollen gel beads. Molecules greater than a certain size do not enter the gel beads and thus move through the chromatographic bed the fastest. Smaller molecules, such as detergent, protein, DNA and the like, which enter the gel beads to varying extent depending on their size and shape, are retarded in their passage through the bed. Molecules are thus generally eluted in the order of decreasing molecular size.

Porous chromatographic resins appropriate for size-exclusion chromatography of viruses may be made of dextrose, agarose, polyacrylamide, or silica which have different physical characteristics. Polymer combinations can also be also used. Most commonly used are those under the tradename, “SEPHADEX” available from Amersham Biosciences. Other size exclusion supports from different materials of construction are also appropriate, for example Toyopearl 55F (polymethacrylate, from Tosoh Bioscience, Montgomery Pa.) and Bio-Gel P-30 Fine (BioRad Laboratories, Hercules, Calif.).

In some exemplary embodiments, SEC can be operated in “desalting mode” to achieve online buffer exchange prior to native MS detection. Desalting fulfills the goal of removing buffer salts from a sample in exchange for water (with water used to pre-equilibrate the SEC resin). A desalting SEC method suitable for the method of the present invention is described in, for example, Yan et al., 2020, J Am Soc Mass Spectrom, 31:2171-2179. Desalting SEC allows for the subsequent MS analysis of samples eluted from cIEF, which otherwise may have incompatible buffer conditions.

The protein load of a sample comprising a protein of interest can be adjusted to a total protein load to the column of between about 50 g/L and about 1000 g/L; about 5 g/L and about 150 g/L, between about 10 g/L and about 100 g/L, between about 20 g/L and about 80 g/L, between about 30 g/L and about 50 g/L, or between about 40 g/L and about 50 g/L. In certain embodiments, the protein concentration of the load protein mixture is adjusted to a protein concentration of the material to be loaded onto the column of between about 0.5 g/L and about 50 g/L, or between about 1 g/L and about 20 g/L.

As used herein, the term “mass spectrometer” includes a device capable of identifying specific molecular species and measuring their accurate masses. The term is meant to include any molecular detector into which a polypeptide or peptide may be characterized. A mass spectrometer can include three major parts: the ion source, the mass analyzer, and the detector. The role of the ion source is to create gas phase ions. Analyte atoms, molecules, or clusters can be transferred into gas phase and ionized either concurrently (as in electrospray ionization) or through separate processes. The choice of ion source depends on the application. In some exemplary embodiments, the mass spectrometer can be a tandem mass spectrometer. As used herein, the term “tandem mass spectrometry” includes a technique where structural information on sample molecules is obtained by using multiple stages of mass selection and mass separation. A prerequisite is that the sample molecules be transformed into a gas phase and ionized so that fragments are formed in a predictable and controllable fashion after the first mass selection step. Multistage MS/MS, or MSⁿ, can be performed by first selecting and isolating a precursor ion (MS²), fragmenting it, isolating a primary fragment ion (MS³), fragmenting it, isolating a secondary fragment (MS⁴), and so on, as long as one can obtain meaningful information, or the fragment ion signal is detectable. Tandem MS has been successfully performed with a wide variety of analyzer combinations. Which analyzers to combine for a certain application can be determined by many different factors, such as sensitivity, selectivity, and speed, but also size, cost, and availability. The two major categories of tandem MS methods are tandem-in-space and tandem-in-time, but there are also hybrids where tandem-in-time analyzers are coupled in space or with tandem-in-space analyzers. A tandem-in-space mass spectrometer comprises an ion source, a precursor ion activation device, and at least two non-trapping mass analyzers. Specific m/z separation functions can be designed so that in one section of the instrument ions are selected, dissociated in an intermediate region, and the product ions are then transmitted to another analyzer for m/z separation and data acquisition. In tandem-in-time, mass spectrometer ions produced in the ion source can be trapped, isolated, fragmented, and m/z separated in the same physical device. The peptides identified by the mass spectrometer can be used as surrogate representatives of the intact protein and their post translational modifications. They can be used for protein characterization by correlating experimental and theoretical MS/MS data, the latter generated from possible peptides in a protein sequence database. The characterization includes, but is not limited, to sequencing amino acids of the protein fragments, determining protein sequencing, determining protein de novo sequencing, locating post-translational modifications, or identifying post translational modifications, or comparability analysis, or combinations thereof.

In some exemplary aspects, the mass spectrometer can work on nanoelectrospray or nanospray.

The term “nanoelectrospray” or “nanospray” as used herein refers to electrospray ionization at a very low solvent flow rate, typically hundreds of nanoliters per minute of sample solution or lower, often without the use of an external solvent delivery. The electrospray infusion setup forming a nanoelectrospray can use a static nanoelectrospray emitter or a dynamic nanoelectrospray emitter. A static nanoelectrospray emitter performs a continuous analysis of small sample (analyte) solution volumes over an extended period of time. A dynamic nanoelectrospray emitter uses a capillary column and a solvent delivery system to perform chromatographic separations on mixtures prior to analysis by the mass spectrometer.

In some exemplary embodiments, SEC-MS can be performed under native conditions.

As used herein, the term “native conditions” can include performing mass spectrometry under conditions that preserve non-covalent interactions in an analyte. For detailed review on native MS, refer to the review: Elisabetta Boeri Erba & Carlo Pe-tosa, The emerging role of native mass spectrometry in characterizing the structure and dynamics of macromolecular complexes, 24 PROTEIN SCIENCE 1176-1192 (2015).

In some exemplary aspects, the mass spectrometer can be a tandem mass spectrometer.

As used herein, the term “tandem mass spectrometry” includes a technique where structural information on sample molecules is obtained by using multiple stages of mass selection and mass separation. A prerequisite is that the sample molecules can be transferred into gas phase and ionized intact and that they can be induced to fall apart in some predictable and controllable fashion after the first mass selection step. Multistage MS/MS, or MSⁿ, can be performed by first selecting and isolating a precursor ion (MS²), fragmenting it, isolating a primary fragment ion (MS), fragmenting it, isolating a secondary fragment (MS⁴), and so on as long as one can obtain meaningful information, or the fragment ion signal is detectable. Tandem MS has been successfully performed with a wide variety of analyzer combinations. What analyzers to combine for a certain application can be determined by many different factors, such as sensitivity, selectivity, and speed, but also size, cost, and availability. The two major categories of tandem MS methods are tandem-in-space and tandem-in-time, but there are also hybrids where tandem-in-time analyzers are coupled in space or with tandem-in-space analyzers. A tandem-in-space mass spectrometer comprises an ion source, a precursor ion activation device, and at least two non-trapping mass analyzers. Specific m z separation functions can be designed so that in one section of the instrument ions are selected, dissociated in an intermediate region, and the product ions are then transmitted to another analyzer for m z separation and data acquisition. In tandem-in-time, mass spectrometer ions produced in the ion source can be trapped, isolated, fragmented, and m z separated in the same physical device.

The peptides identified by the mass spectrometer can be used as surrogate representatives of the intact protein and their post-translational modifications. They can be used for protein characterization by correlating experimental and theoretical MS/MS data, the latter generated from possible peptides in a protein sequence database. The characterization includes, but is not limited, to sequencing amino acids of the protein fragments, determining protein sequencing, determining protein de novo sequencing, locating post-translational modifications, or identifying post-translational modifications, or comparability analysis, or combinations thereof.

As used herein, the term “database” refers to a compiled collection of protein sequences that may possibly exist in a sample, for example in the form of a file in a FASTA format. Relevant protein sequences may be derived from cDNA sequences of a species being studied. Public databases that may be used to search for relevant protein sequences included databases hosted by, for example, Uniprot or Swiss-prot. Databases may be searched using what are herein referred to as “bioinformatics tools”. Bioinformatics tools provide the capacity to search uninterpreted MS/MS spectra against all possible sequences in the database(s), and provide interpreted (annotated) MS/MS spectra as an output. Non-limiting examples of such tools are Mascot (www.matrixscience.com), Spectrum Mill (www.chem.agilent.com), PLGS (www.waters.com), PEAKS (www.bioinformaticssolutions.com), Proteinpilot (download.appliedbiosystems.com//proteinpilot), Phenyx (www.phenyx-ms.com), Sorcerer (www.sagenresearch.com), OMSSA (www.pubchem.ncbi.nlm.nih.gov/omssa/), X!Tandem (www.thegpm.org/TANDEMI), Protein Prospector (prospector.ucsf.edu/prospector/mshome.htm), Byonic (www.proteinmetrics.com/products/byonic) or Sequest (fields.scripps.edu/sequest).

In some exemplary embodiments, the mass spectrometer is coupled to the chromatography system, for example, SEC or desalting SEC.

It is understood that the present invention is not limited to any of the aforesaid protein(s), antibody(s), pI(s), protein alkylating agent(s), protein denaturing agent(s), protein reducing agent(s), digestive enzyme(s), hydrolyzing agent(s), charge variant(s), post-translational modification(s), sample(s), IEF system(s), SEC system(s), mass spectrometer(s), database(s), or bioinformatics tool(s), and any protein(s), antibody(s), pI(s), protein alkylating agent(s), protein denaturing agent(s), protein reducing agent(s), digestive enzyme(s), hydrolyzing agent(s), charge variant(s), post-translational modification(s), sample(s), IEF system(s), SEC system(s), mass spectrometer(s), database(s), or bioinformatics tool(s) can be selected by any suitable means.

The present invention will be more fully understood by reference to the following Examples. They should not, however, be construed as limiting the scope of the invention.

### EXAMPLES

### Example 1. Overview of the Method of the Present Invention

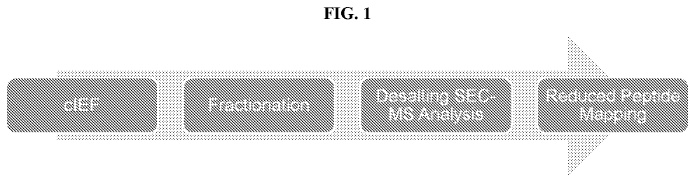

A novel method for characterizing charge variants of a protein of interest is disclosed herein. An exemplary workflow for the method of the present invention is shown in FIG. 1. A sample, for example a sample from a production step of a therapeutic protein product or any protein of interest, is subjected to capillary isoelectric focusing (cIEF). cIEF separates components of the protein sample based on charge (pI), including separating charge variants of the protein of interest. A UV trace of sample components traversing the cIEF capillary is generated, with local maxima of protein concentration considered “peaks,” and corresponding to protein charge variants. Fractions are serially collected from the capillary. Fractions may be collected in a high-throughput fashion such that all of the cIEF eluate is collected, and fractions represent narrow intervals, for example, 15 second intervals. This high-throughput fraction collection allows for the preservation of the high-resolution separation of cIEF, without requiring an online connection to a mass spectrometer for later MS analysis.

Optionally, fractions may be contacted to hydrolyzing agents, alkylating agents and/or reducing agents to generate reduced peptide fragments of the protein of interest. Fractions may be recombined prior to subsequent analysis depending on the desired concentration and resolution of the analysis.

Fractions are then subjected to desalting size exclusion chromatography (SEC). Desalting SEC serves dual purposes: exchanging the buffer from the collected cIEF fractions with a buffer that is compatible with mass spectrometry (MS) analysis, and further separating fraction components based on size.

Finally, eluate from desalting SEC is subjected to mass spectrometry analysis, for example, intact mass analysis or reduced peptide mapping analysis. The desalting SEC may be connected online with a mass spectrometer (desalting SEC-MS). In particular, reduced peptide mapping analysis allows for the identification of site-specific protein modifications that may contribute to charge variation. Because the identified protein modifications arise from a known fraction, and fractions coincide with known portions of the cIEF UV trace, a causal relationship can be drawn between site-specific protein modifications and the protein charge variants.

### Example 2. Intact Mass Analysis of Protein Charge Variants

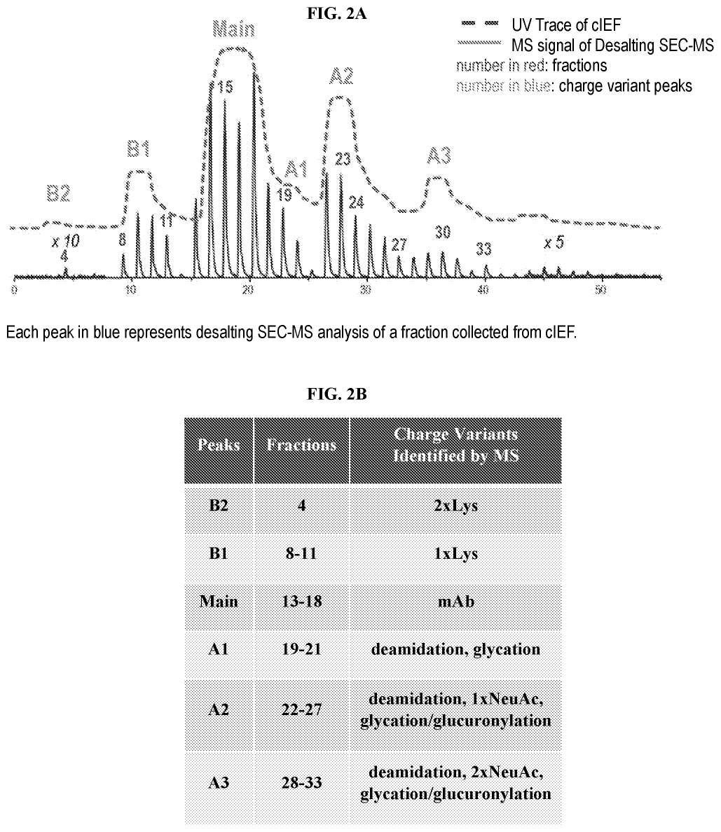

Using the method of the present invention, a bispecific antibody, bsAb-1, was subjected to the steps of cIEF, fractionation, and desalting SEC-MS. The UV trace generated by cIEF (shown in red) and MS signal generated by desalting SEC-MS (shown in blue) can be correlated as shown in FIG. 2A. Six UV peaks (corresponding to charge variants) were designated for this UV trace: B2, B1, Main, A1, A2, and A3 (ordered from basic to acidic). Each blue peak represents a desalting SEC-MS analysis of a fraction.

Desalting SEC-MS analysis of cIEF fractions allows for assignment of specific protein modifications to charge variants, as shown in FIG. 2B. Because each desalting SEC-MS analysis arises from a known fraction, and each fraction represents a known portion of the UV trace, protein modifications detected by MS can be directly associated with charge variants detected by cIEF. In this fashion, the specific causes of charge variants of a protein of interest can be uncovered.

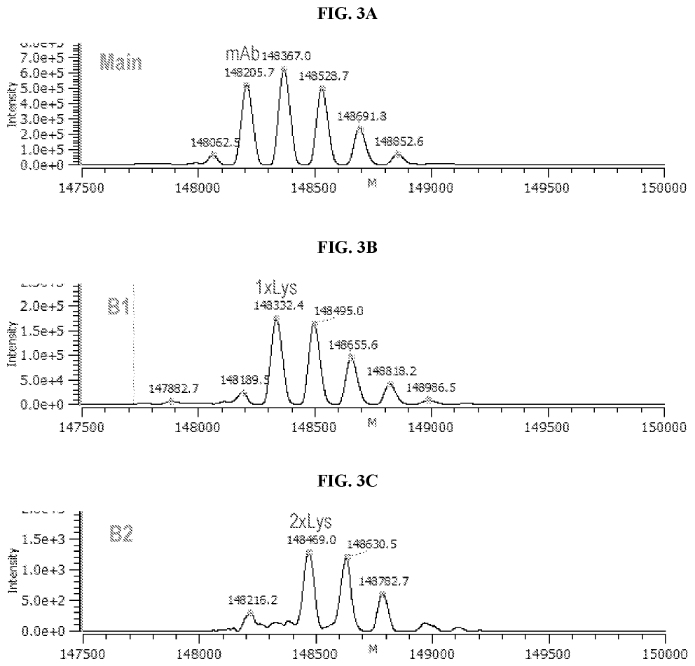

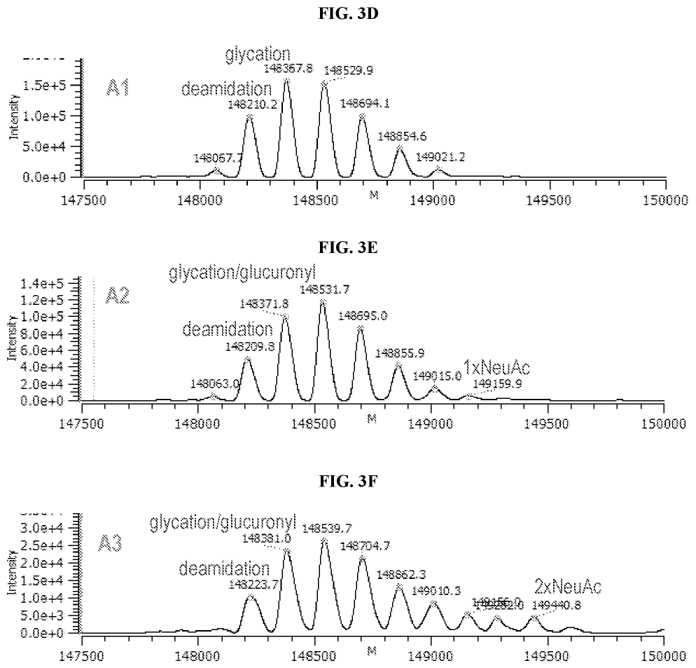

Further analysis is shown in FIG. 3. Deconvoluted mass spectra for each charge variant of bsAb-1 detected by desalting SEC-MS are demonstrated, with each featuring various m z peaks that may be identified with specific protein modifications. FIG. 3A shows the main charge variant and a peak representing the main mAb species. FIG. 3B shows the B1 charge variant and a peak representing a species with a C-terminal lysine. FIG. 3C shows the B2 charge variant and a peak representing a species with two C-terminal lysines. FIG. 3D shows the A1 charge variant and one peak representing a species with a glycation modification, and one peak representing a species with a deamidation modification. FIG. 3E shows the A2 charge variant and one peak representing a species with a glycation/glucuronyl modification, one peak representing a species with a deamidation modification, and one peak representing a species with a N-acetylneuraminic acid modification. FIG. 3F shows the A3 charge variant and one peak representing a species with a glycation/glucuronyl modification, one peak representing a species with a deamidation modification, and one peak representing a species with two N-acetylneuraminic acid modifications.

These experiments demonstrate the capacity of the method of the present invention to use desalting SEC-MS to determine the specific protein modifications corresponding to and causative of charge variants detected by cIEF.

### Example 3. Reduced Peptide Mapping Analysis of Protein Charge Variants

The method of the present invention can also be used with reduced peptide mapping analysis to identify protein modifications at specific residues of a protein of interest, and associate these site-specific modifications with charge variants of the protein. A bispecific antibody, bsAb-1, was subjected to the steps of cIEF, fractionation, and desalting SEC-MS as previously described. Fractions were subjected to protein reduction and hydrolysis prior to desalting SEC in order to produce reduced peptide fragments.

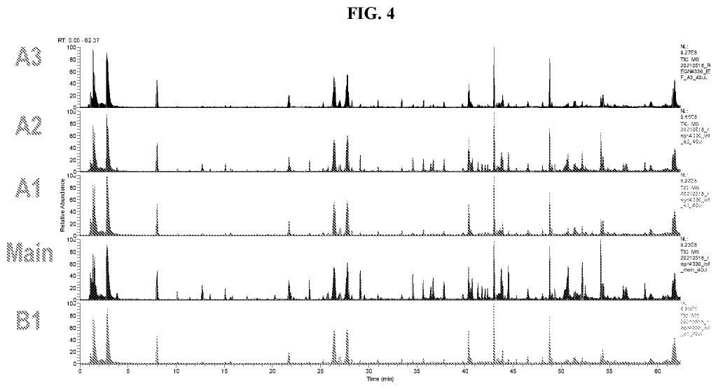

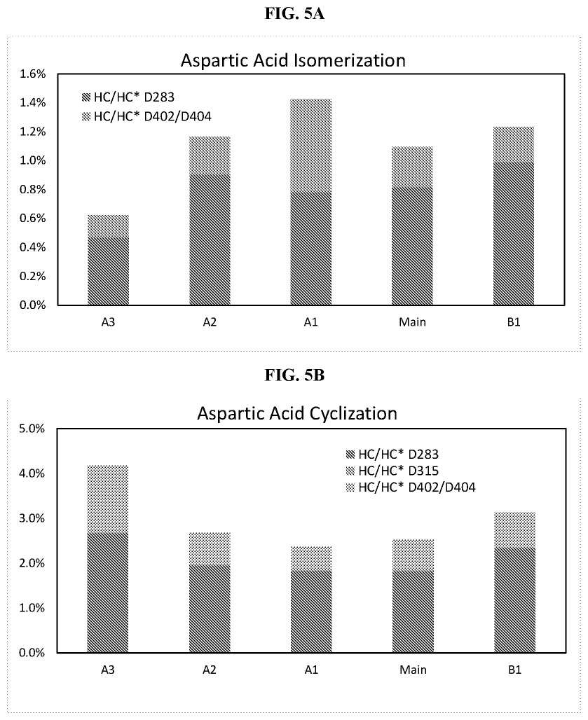

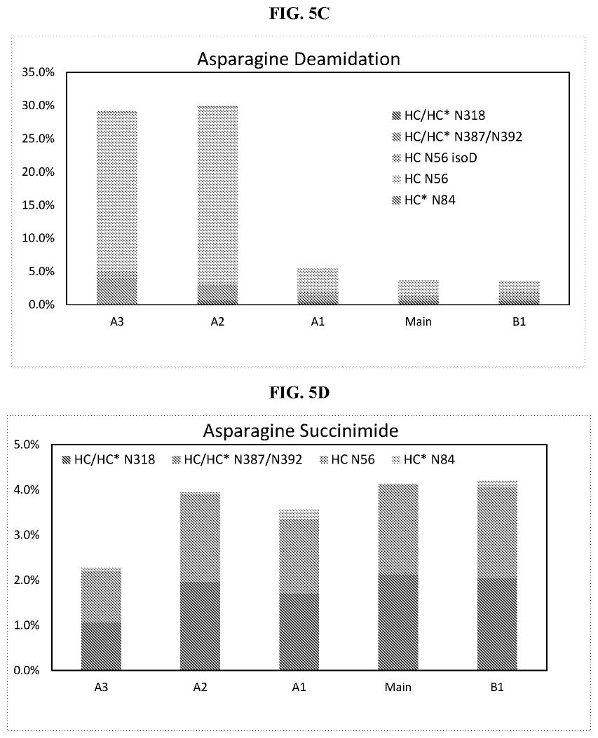

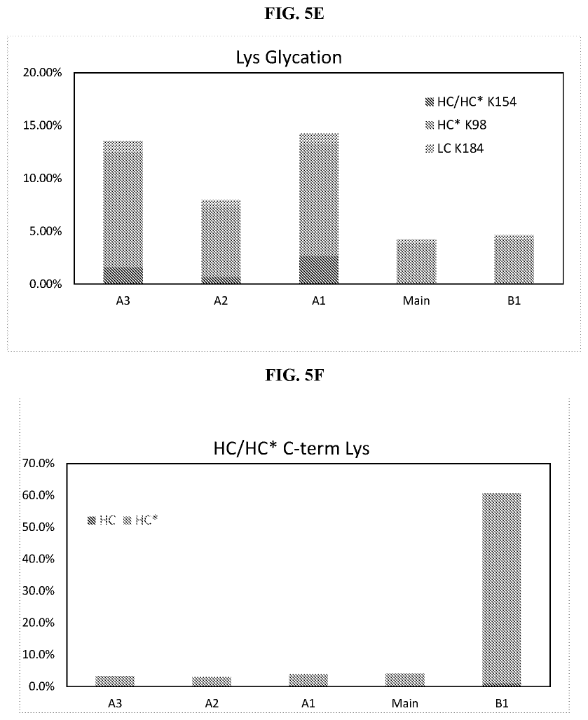

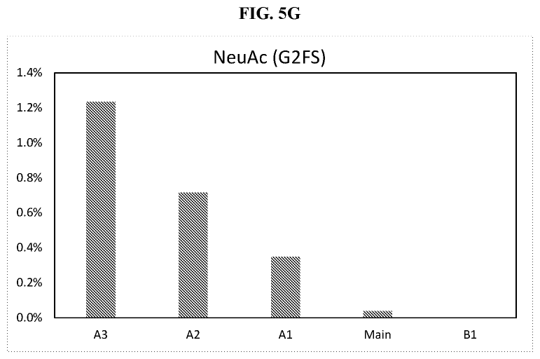

Exemplary MS signal corresponding to each charge variant detected by cIEF is shown in FIG. 4. Peptides were analyzed using peptide mapping, and specific protein modifications at specific residues were identified as shown in FIG. 5. Using this method, the statistical distribution across charge variants of protein modifications at specific amino acid residues could be established. FIGS. 5A-5G demonstrate exemplary modifications that can lead to charge variants, including aspartic acid isomerization, aspartic acid cyclization, asparagine deamidation, asparagine succinimides, lysine glycation, C-terminal lysines, or N-acetylneuraminic acids. Specific modified residues are identified across the light chain (LC), first heavy chain (HC) or second heavy chain (HC*) of bsAb-1.

These experiments demonstrate the capacity of the method of the present invention to provide amino acid residue level-resolution about specific protein modifications that may cause specific charge variants of a protein of interest. This information can be used, for example, to monitor and/or modify the production process of a therapeutic protein in order to achieve acceptable biophysical properties and product homogeneity.

What is claimed is:

1. A method for characterizing charge variants of a protein of interest comprising:
(a) subjecting a sample including a protein of interest to capillary isoelectric focusing to separate charge variants of said protein of interest;
(b) collecting fractions from said capillary isoelectric focusing step;
(c) subjecting said fractions to desalting size exclusion chromatography; and
(d) subjecting the eluate from step (c) to mass spectrometry analysis to characterize said charge variants of said protein of interest.

2. The method of claim 1, wherein said protein is an antibody, a bispecific antibody, a monoclonal antibody, a fusion protein, an antibody-drug conjugate, an antibody fragment, or a protein pharmaceutical product.

3. The method of claim 1, wherein said capillary isoelectric focusing is imaged capillary isoelectric focusing.

4. The method of claim 1, wherein said desalting size exclusion chromatography system is coupled to said mass spectrometer.

5. The method of claim 1, wherein said mass spectrometer is an electrospray ionization mass spectrometer, nano-electrospray ionization mass spectrometer, or a triple quadrupole mass spectrometer.

6. The method of claim 1, wherein said mass spectrometry analysis comprises intact mass analysis or reduced peptide mapping analysis.

7. The method of claim 1, wherein said mass spectrometer is capable performing a multiple reaction monitoring or parallel reaction monitoring.

8. The method of claim 1, further comprising a step wherein said fractions are contacted to at least one hydrolyzing agent prior to desalting size exclusion chromatography.

9. The method of claim 8, wherein said at least one hydrolyzing agent is chosen from a group consisting of trypsin, chymotrypsin, LysC, LysN, AspN, GluC and ArgC.

10. The method of claim 1, further comprising a step wherein said fractions are contacted to at least one reducing agent prior to desalting size-exclusion chromatography.

11. The method of claim 1, wherein said desalting size exclusion chromatography is performed under native conditions.
